(19) United States
(12) Patent Application Publication
(10) Pub. No.: US 2015/0065444 A1
(43) Pub. Date: Mar. 5, 2015

(54) METHOD FOR ENHANCING EFFICACY AND SELECTIVITY OF CANCER CELL KILLING BY DNA DAMAGING AGENTS

(71) Applicant: EASTERN MAINE HEALTHCARE SYSTEMS, Brewer, ME (US)

(72) Inventor: Derek J. HOELZ, Bangor, ME (US)

(21) Appl. No.: 14/454,496

(22) Filed: Aug. 7, 2014

Related U.S. Application Data
(63) Continuation of application No. 13/407,442, filed on Feb. 28, 2012, now abandoned.
(60) Provisional application No. 61/447,384, filed on Feb. 28, 2011.

(51) Int. Cl. A61K 31/713; A61K 31/704

(52) U.S. Cl. CPC A61K 31/713; A61K 31/704
USPC 514/34

(57) ABSTRACT

The invention relates to the treatment of cancer using DNA damaging agents. The invention provides methods for treating a mammal with cancer, the method comprising inhibiting in the mammal acidic residue methyltransferase (Arm1) in combination with administering to the mammal a DNA damaging agent. The invention further provides pharmaceutical formulations comprising an inhibitor of acidic residue methyltransferase (Arm1) and a DNA damaging agent.

### STATEMENT OF GOVERNMENT SUPPORT

This work was supported by startup funds (D.J.H.) from Eastern Maine Healthcare Systems, by the US Army Medical Research and Materiel Command Contract W81XWH-10-2-0014. The government has certain rights in the invention.

### BACKGROUND OF THE INVENTION

1. Field of the Invention

The invention relates to the use of DNA damaging agents for the treatment of cancer.

2. Summary of the Related Art

DNA damaging agents, such as doxorubicin, have been widely used in the treatment of cancer. Such agents selectively kill proliferating cells while being less toxic to non-proliferating cells, thus providing some measure of cancer cell selectivity, since most cells of the body are non-proliferating. However, important normal cell types, such as intestinal endothelium, immune system cells, bone marrow cells and hair follicle cells do proliferate, and thus are also killed by DNA damaging agents, leading to numerous unwanted side effects. There is, therefore a need to improve the efficacy and selectivity of DNA damaging agents for the treatment of cancer.

### BRIEF SUMMARY OF THE INVENTION

The invention relates to the treatment of cancer using DNA damaging agents. The inventor has surprisingly discovered that knockdown of a previously uncharacterized gene, acidic residue methyltransferase (Arm1), improves the ability of cells having a wild-type p53 gene to survive treatment with DNA damaging agents, while causing cells having mutant p53 genes to become more sensitive to killing by DNA damaging agents. Since more than 50% of cancer cell types have mutant p53 genes, while normal proliferating cells have wild type p53 genes, inhibition of Arm1 increases both the efficacy and selectivity of DNA damaging agents for killing cancer cells.

In a first aspect, the invention provides a method for treating a mammal with cancer, the method comprising inhibiting in the mammal acidic residue methyltransferase (Arm1) in combination with administering to the mammal a DNA damaging agent.

In a second aspect, the invention provides a pharmaceutical formulation comprising an inhibitor of acidic residue methyltransferase (Arm1) and a DNA damaging agent.

### BRIEF DESCRIPTION OF THE DRAWINGS

FIG. 1a shows that a carboxyl methyltransferase targets PCNA in MDA MB 468 cells. FIG. 1b shows that SAM-dependent methyltransferase domains exist in the C6orf211 protein (SEQ ID NOS: 1-8, respectively). FIG. 1c shows the positions of the motifs I, II and regions II and III in CheR and the C6orf211 protein. FIGS. 1d and 1e show an illustrative representation of the SAM-MT fold in the C-terminus of the CheR. Figure if shows recombinant 6× His-tagged Arm1 isolated from Tni insect cell extracts and analyzed by SDS-PAGE and colloidal Coomassie Blue staining FIG. 1g shows that Arm possesses a PCNA-dependent carboxyl methyltransferase activity.

FIG. 2a shows that Doxorubicin (Dox) promotes PCNA methyl esterification in MCF7 cells. FIG. 2b shows that PCNA-dependent methyltransferase activity is altered following Dox treatment. FIG. 2c shows that Dox induces p21 expression in MCF7 cells. FIG. 2d shows that p21 binding promotes PCNA methyl esterification. FIG. 2e shows that the p21-induced basic shift is a result of PCNA methyl esterification. FIG. 2f shows that p21 does not interact with Arm1. FIG. 2g shows that PCNA does not directly interact with Arm1 in SK-Br-3 cells.

FIG. 3a shows that PCNA-dependent methyltransferase activities are altered in shRNA expressing SK-Br-3 and MCF7 cells. FIG. 3b shows that DNA damage sensitivity in Arm1 knockdown cells is related to p53 status. FIG. 3c shows that Arm1 knockdown promotes DNA repair.

FIG. 4a shows that PCNA chromatin stability is unaffected by Arm1. FIG. 4b shows that Arm1 is recruited to the chromatin and promotes PCNA ubiquitylation. FIG. 4c shows that Arm1-dependent methyltransferase activity promotes Rad18's interactions with Arm1 and PCNA. FIG. 4d shows that Arm1 interacts with Rev1.

FIGS. 5a and b show a model for PCNA methyl esterification.

FIG. 6a shows fractions from passage over a Superdex S200 gel filtration column. FIG. 6b shows active fractions resolved by 2D-PAGE and stained with colloidal Coomassie Blue. The position of the 50 kDa product of the uncharacterized gene C6orf211 is identified with an arrow. FIG. 6c shows proteins present in the enriched fractions identified by mass spectrometry and grouped by cellular function.

FIG. 7 (SEQ ID NOS: 9-11, respectively) shows sequence alignments of the CheR, C6orf211, and PIMT proteins. Amino acids are colour coded green (polar), red (nonpolar, hydrophobic), pink (basic), and blue (acidic).

FIG. 8 (SEQ ID NOS: 12-19, respectively) shows alignment of the C6orf211 proteins from eight eukaryotic organisms with motifs I and II and regions II and III identified.

FIG. 9 (SEQ ID NOS: 20-25, respectively) shows patterns of methyl esterification of peptides from p21-PIP affinity purified PCNA isoforms separated and excised from 2D-PAGE gels and analyzed by LC-MS/MS. Positively identified peptide sequences are shown in black and unobserved sequences are shown in red. The locations of methyl esterified residues in the PCNA isoform spots are presented in bolded blue.

FIG. 10a shows shRNA expression. FIG. 10b shows reduction of Arm1 mRNA expression in shRNA expressing cells.

### DETAILED DESCRIPTION OF THE PREFERRED EMBODIMENTS

The invention relates to the treatment of cancer using DNA damaging agents. The inventor has surprisingly discovered that knockdown of a previously uncharacterized gene, acidic residue methyltransferase (Arm1), improves the ability of cells having a wild-type p53 gene to survive treatment with DNA damaging agents, while causing cells having mutant p53 genes to become more sensitive to killing by DNA damaging agents. Since more than 50% of cancer cell types have mutant p53 genes, while normal proliferating cells have wild type p53 genes, inhibition of Arm1 increases both the efficacy and selectivity of DNA damaging agents for killing cancer cells.

In a first aspect, the invention provides a method for treating a mammal with cancer, the method comprising inhibiting in the mammal acidic residue methyltransferase (Arm1) in combination with administering to the mammal a DNA damaging agent.

“Treating a mammal with cancer” means causing in the mammal a reduction of signs or symptoms of cancer.

“Inhibiting acidic residue methyltransferase 1 (Arm1)” means reducing the activity and/or expression of Arm1. Preferred methods of inhibiting Arm1 include, without limitation, contacting a cancer cell with a small molecule inhibitor of Arm1 activity, or a dominant negative mutant of Arm1, such as an Arm1 protein with some but not all of its protein- or substrate-interactive domains inactivated or a genetic suppressor element (GSE) that encodes a fragment of the Arm1 protein, which interferes with the Arm1 activity. Contacting a tumor cell with a dominant negative mutant of Arm1 includes expressing the dominant negative mutant via transfection with a virus or a vector expressing the dominant negative mutant, or contacting a cancer cell with a peptide encoded by the GSE. Additional preferred methods include contacting a cell with an inhibitor of Arm1 gene expression, including without limitation, a short hairpin RNA (shRNA), a small inhibitory RNA (siRNA), an antisense nucleic acid (AS) and a ribozyme. “Contacting a tumor cell with an inhibitor of Arm1 gene expression” includes exogenously providing to a cell an inhibitor of Arm1 gene expression, as well as expressing an inhibitor of Arm1 gene expression in a cell. Expressing an inhibitor of gene expression in a cell is conveniently provided by transfection with a virus or a vector expressing such an inhibitor.

“Administering to the mammal a DNA damaging agent” means providing the mammal with a DNA damaging agent by any medically acceptable route, including, without limitation, parenteral, oral, sublingual, transdermal, topical, intranasal, intratracheal, or intrarectal. In certain preferred embodiments, compositions of the invention are administered parenterally, e.g., intravenously in a hospital setting. In certain other preferred embodiments, administration may preferably be by the oral route. Preferred DNA damaging agents include, without limitation, doxorubicin, 6-mercaptopurine, Gemcitabine, Cyclophosphamide, Melphalan, Busulfan, Chlorambucil, Mitomycin, Cisplatin, Bleomycin, Dectinomycin, Irinotecan and Mitoxantrane.

In combination with means in the course of treating the same disease in the same mammal, and includes inhibiting Arm1 and administering the DNA damaging agent in any order, including simultaneous administration, as well as any temporally spaced order, for example, from sequentially with one immediately following the other to up to several hours apart. The administration of an inhibitor of Arm1 and DNA damaging agent may be by the same or different routes.

In the methods for treatment according to the invention, the compounds and other inhibitors described above may be incorporated into a pharmaceutical formulation. Such formulations comprise the compound, which may be in the form of a free acid, salt or prodrug, in a pharmaceutically acceptable diluent, carrier, or excipient. Such formulations are well known in the art and are described, e.g., in Remington's Pharmaceutical Sciences, 18th Edition, ed. A. Gennaro, Mack Publishing Co., Easton, Pa., 1990.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art.

As used herein, the term “pharmaceutically acceptable salts” refers to salts that retain the desired biological activity of the above-identified compounds and exhibit minimal or no undesired toxicological effects. Examples of such salts include, but are not limited to, salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, palmoic acid, alginic acid, polyglutamic acid, naphthalenesulfonic acid, naphthalenedisulfonic acid, methanesulfonic acid, p-toluenesulfonic acid, polygalacturonic acid, and the like. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

In a second aspect, the invention provides a pharmaceutical formulation comprising an inhibitor of acidic residue methyltransferase (Arm1) and a DNA damaging agent. Preferred inhibitors of Arm1 include, without limitation, small molecule inhibitors of Arm1 activity, dominant negative mutants of Arm1, such as an Arm1 protein with some but not all of its protein- or substrate-interactive domains inactivated, genetic suppressor elements (GSEs) that encodes a fragment of the Arm1 protein, which interferes with the Arm1 activity.

Preferred DNA damaging agents include, without limitation, doxorubicin, 6-mercaptopurine, Gemcitabine, Cyclophosphamide, Melphalan, Busulfan, Chlorambucil, Mitomycin, Cisplatin, Bleomycin, Dectinomycin, Irinotecan and Mitoxantrane.

The pharmaceutical formulation may further comprise additional diluents, excipients or carriers, as described above for the first aspect of the invention.

The following examples are intended to further illustrate certain preferred embodiments of the invention and are not to be construed as limiting the scope of the invention.

### Example 1

### Cell Culture

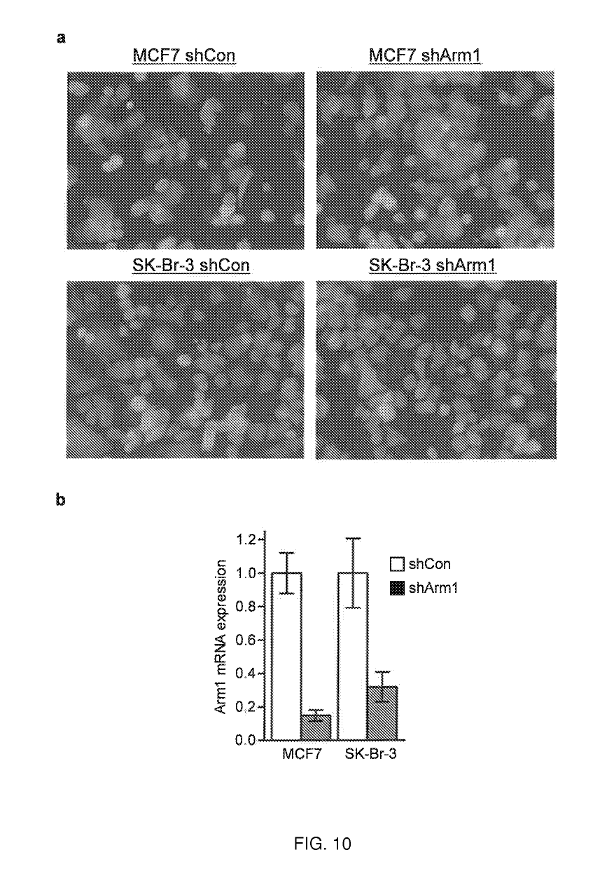

MCF7 and SK-Br-3 cells were obtained from ATCC and maintained in DMEM or McCoys 5A supplemented with 10% FBS and antibiotics at 37° C., 5% CO₂. A human Flag-tagged Arm1, Rev1, and p21 expression construct (Origene) were transiently transfected into SK-Br-3 cells with Fugene 6 (Roche) and extracts generated after 24 h. Lentiviral shRNA particles were obtained from Open Biosystems and stably expressing clones selected with puromycin and confirmed by GFP expression and Q-PCR (FIG. 10). Clonogenic survival assays were performed following exposure to Dox (Sigma) or UV-C using a Spectrolinker (Spectronics) for 4 h. Surviving colonies were stained with methylene blue and counted 2 weeks after treatments.

### Example 2

### Vapour Diffusion Assay

The assay was performed as previously described.³⁷ Cell extracts were assayed with [³H-methyl]-SAM (NEN) for 1 h before equilibration with 100 mM NaOH with 1% SDS and spotting onto filter paper folded into an accordion pleat and placed above scintillation fluid. Diffused ³H-methanol was detected the following day.

### Example 3

### Protein Expression and Purification

Chromatography was performed using a Biologic DuoFlow (BioRad) using phenyl Sepharose HP (HiTrap) and Superdex S200 columns (GE Biosciences). Recombinant PCNA was expressed either as a calmodulin binding peptide fusion (^{CBP}PCNA) using the pDual expression system and purified using calmodulin agarose (Stratagene) or a 6× His-tagged fusion expressed in pET303/CT-His (InVitrogen) and purified with Ni²⁺ Sepharose (GE Biosciences). His-tagged human Arm1 was cloned into a baculovirus expression vector and expressed in Tni insect cells (Allele Biotech., Inc.). GST, GST-p21, GST-p21(PIP), and GST-Rad18 were expressed in BL21(DE3) cells and isolated using glutathione Sepharose (GE Biosciences). GST-p21 was isolated from inclusion bodies as described³⁸. Anti-Flag immunoprecipitations were performed with anti-Flag M2 Affinity Gel (Sigma). p21(PIP)-affinity beads were generated by covalently coupling a synthetic peptide (Anaspec) to CH-Sepharose (GE Biosciences).

### Example 4

### Electrophoresis and Mass Spectrometry

2D-PAGE and protein identification and sequencing by LC-MS/MS was performed as previously described¹⁰. Anti-PCNA (PC10) were from Millipore, anti-histone H3 were from Cell Signalling, anti-p21 (C19) and anti-α-tubulin antibodies were from Santa Cruz Biotech, anti-DDK (Flag) antibodies were from Origene, anti-C6orf211 antibodies were from Sigma, and anti-Rad18 was from ThermoElectron.

### Example 5

### C6orf211 Encodes a PCNA-Dependent Carboxyl Methyltransferase (Arm1)

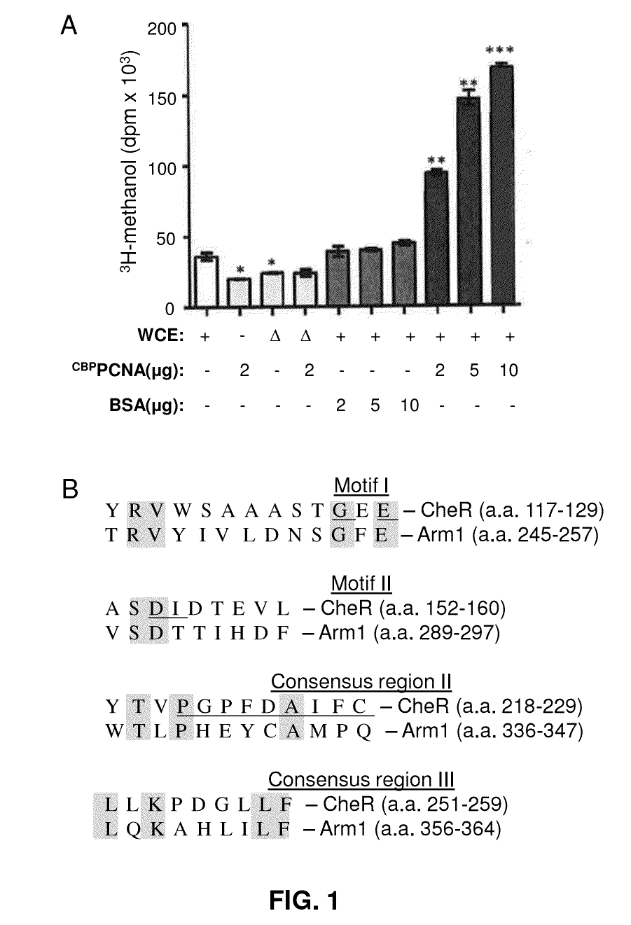

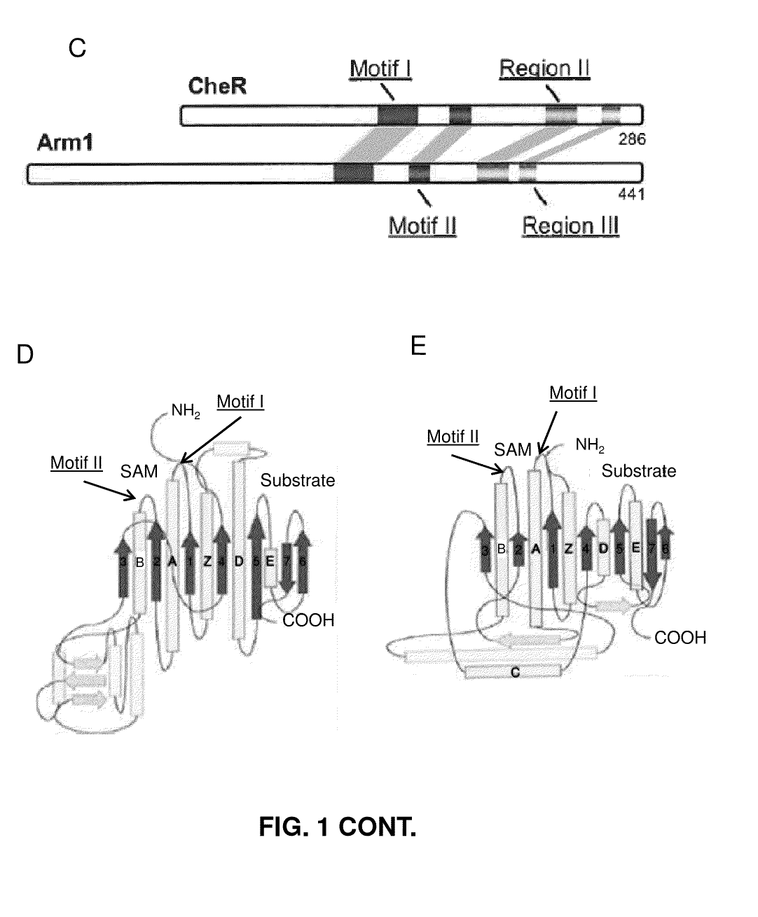

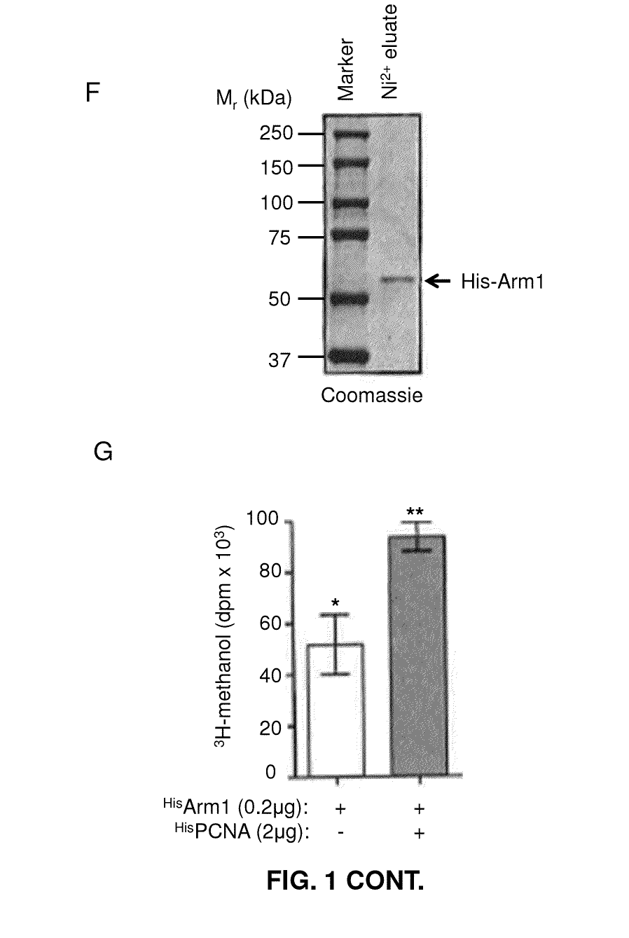

The results from this example are shown in FIG. 1. a, A carboxyl methyltransferase targets PCNA in MDA MB 468 cells. SAM-dependent carboxyl methyltransferase activities of whole cell extracts (WCE) were detected by vapour diffusion assay³⁷. Average activities are presented (±S.E.M). D heat denatured (95° C., 5 min); CBP-PCNA, calmodulin binding peptide (CBP)-tagged PCNA; bovine serum albumin (BSA). Significance was determined using an unpaired two-tailed t-test. b, SAM-dependent methyltransferase domains exist in the C6orf211 protein. Bacterial CheR methyltransferase sequences containing motifs I and II and region III were aligned to the full-length C6orf211 protein sequence using KALIGN³⁹ and align sequences shown. Conserved glycine and glutamic acid residues in CheR motif I and catalytic aspartic acid and conserved isoleucine residues of motif II are underlined. Full-length CheR and C6orf211 protein sequences were aligned with Nomad⁴⁰. CheR's region II sequence is underlined. c, The positions of motifs I, II and regions II and III in CheR and the C6orf211 protein. d, Illustrative representation of the SAM-MT fold in the C-terminus of the CheR¹³. e, The C-terminus of the C6orf211 protein (a.a. 227-441) has the potential for a SAM-MT fold. Secondary structures predicted with Jpred⁴¹ were assembled into a hypothetical SAM-MT fold. Conserved a-helices (A-E) are shown in yellow and b-sheets (1-7) are in magenta. The CheR structure lacks a-helix C. The positions of motifs I and II in CheR and Arm1 structures are highlighted in red and blue, respectively. f, Recombinant 6× His-tagged Arm1 was isolated from Tni insect cell extracts and analyzed by SDS-PAGE and colloidal Coomassie Blue staining. g, Arm possesses a PCNA-dependent carboxyl methyltransferase activity. Purified Arm1 was assayed in the absence and presence of purified 6× His-tagged PCNA using the vapour diffusion assay. Background (His-PCNA alone) subtracted counts from three independent experiments are shown (±S.E.M). Significance was determined using a two-tailed t-test.

### Example 6

### PCNA Methyl Esterification is Promoted by DNA Damage

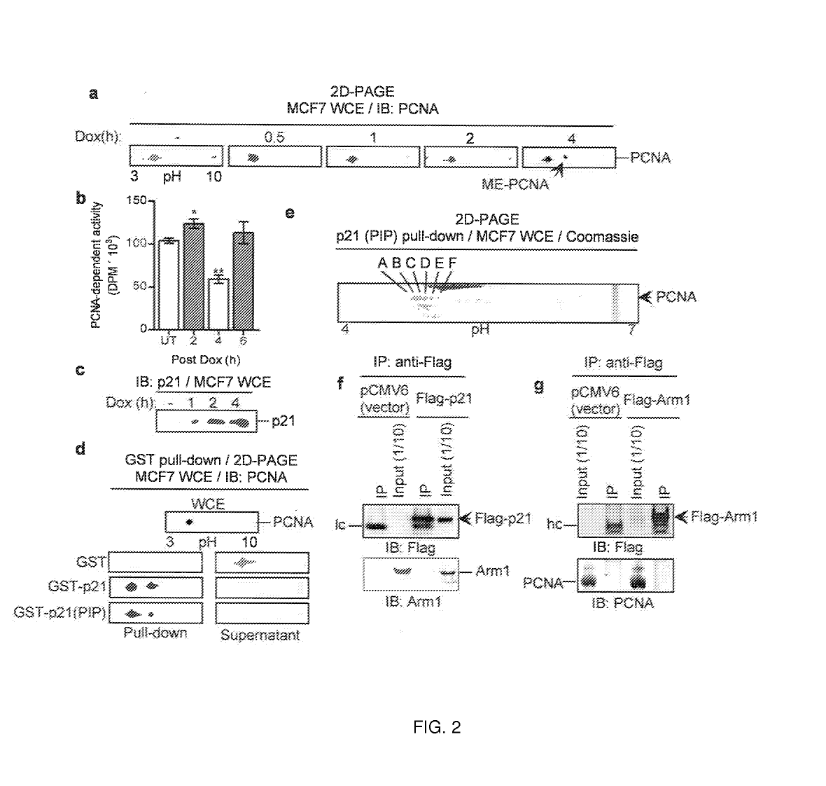

The results from this example are shown in FIG. 2. a, Doxorubicin (Dox) promotes PCNA methyl esterification in MCF7 cells. Whole cell extracts (WCE) from cultures treated with Dox (5 mM) were resolved by 2D-PAGE followed by PCNA immunoblotting (IB). The basic-shifted PCNA isoform (ME-PCNA) is identified with an arrow. b, PCNA-dependent methyltransferase activity is altered following Dox treatment. Dox treated MCF7 WCE were assayed for PCNA-dependent activity in triplicate using the vapour diffusion assay, and average activities are presented ±S.E.M. Significance was determined using a two-tailed t-test. c, Dox induces p21 expression in MCF7 cells. Extracts were separated by SDS-PAGE and immunoblotted for p21^{Waf1/Cip1}. d, p21 binding promotes PCNA methyl esterification. Untreated MCF7 extracts were incubated with GST, GST-p21 (full-length), and GST-p21(PIP) proteins bound to glutathione Sepharose for 2 h at 4° C. prior to 2D-PAGE and PCNA immunoblotting. e The p21-induced basic shift is a result of PCNA methyl esterification. p21(PIP) peptide was covalently coupled to CH-Sepharose and used to affinity purify PCNA from MCF7 whole cell extracts. Purified fractions were separated by 2D-PAGE and stained with colloidal Coomassie blue. Spots were excised from the gel, digested with trypsin, and analyzed by LC-MS/MS. Protein spots identified as PCNA (A-F) were further scrutinized for presence of methyl esters (table I). Methyl esterification of 16 highly conserved glutamate residues and one aspartate residue was observed (FIG. 10). f, p21 does not interact with Arm1. SK-Br-3 cell extracts expressing Flag (vector control) or Flag-p21 were immunoprecipitated with anti-Flag antibodies. Immunoprecipitates were resolved by SDS-PAGE and immunoblotted with anti-Flag and anti-Arm1 antibodies. g, PCNA does not directly interact with Arm1 in SK-Br-3 cells. SK-Br-3 cell extracts expressing Flag (vector control) or Flag-Arm1 were immunoprecipitated with anti-Flag antibodies. Immunoprecipitates were resolved by SDS-PAGE and immunoblotted for Flag and PCNA.

TABLE I
Enrichment of Methyltransferase Activity

| | | | TOTAL | |
|---|---|---|---|---|
| | VOLUME | TOTAL UNITS | PROTEIN | SPECIFIC ACTIVITY |
| PROTEIN FRACTION | (ML) | (FMOL/MIN) | (MG) | (FMOL/MG/MIN) |
| Lysate^{a} | 4 | 58.3 | 20 | 2.9 |
| 30% NH₄SO₄^{a} | 4 | 48 | 12 | 4.0 |
| Phenyl Sepharose HP^{ab} | 5 | 40 | 1.7 | 23.5 |
| Superdex S200^{b} | 1 | 39 | 0.38 | 102.6 |

^{a}Methyl esterification of endogenous proteins was subtracted.
^{b}Activities of pooled peak fractions.

### Example 7

### Arm1-Dependent PCNA Methyl Esterification is Linked to DNA Repair

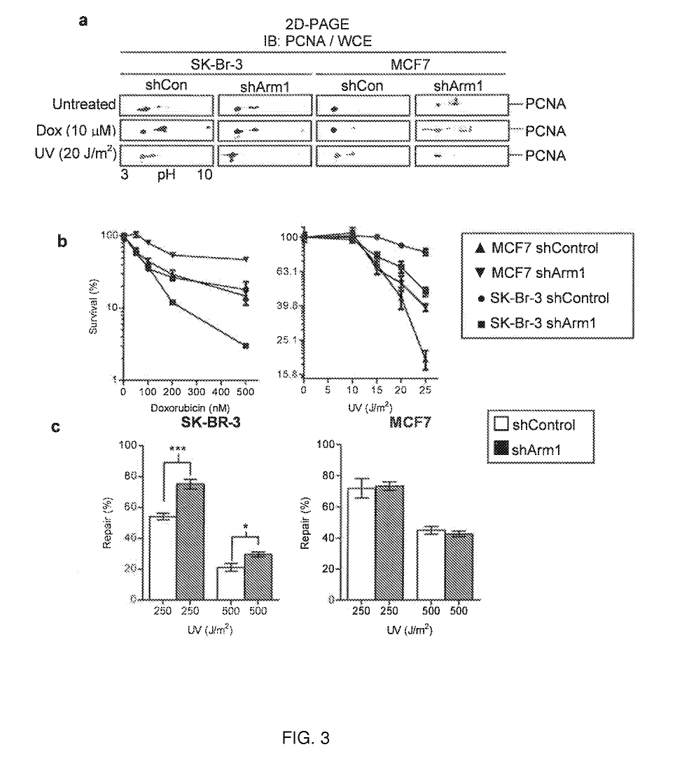

The results from this example are shown in FIG. 3. a, Arm1 promotes PCNA methyl esterification following DNA damage. PCNA was analyzed by 2D-PAGE immunoblotting in SK-Br-3 and MCF7 cells expressing Arm1 or non-targeting (control) shRNA in the absence of DNA damage or 4 h following Dox treatment or UV irradiation. b, PCNA-dependent methyltransferase activities are alter in shRNA expressing SK-Br-3 and MCF7 cells. WCE were assayed for PCNA-dependent carboxyl methyltransferase activity in the presence of His-PCNA (2 mg) and average activities shown (±S.E.M.). Significance was determined using a two-tailed t-test. c, DNA damage sensitivity in Arm1 knockdown cells is related to p53 status. MCF7 (p53 wild-type) and SK-Br-3 (p53-mutant) cells were exposed to increasing levels of Dox and UV and survival determined by clonogenic survival. Average results from three independent experiments are presented ±SD. d, Arm1 knockdown promotes DNA repair. DNA repair rates were determined in shRNA expressing SK-Br-3 and MCF7 cells using host cell reactivation assay. Cells were transfected with UV-irradiated reporter plasmids and average repair rates were determined after 24 h. Average results from independent experiments are presented (±S.E.M.). Significance was determined with a two-tailed t-test.

### Example 8

### Arm1 Promotes DNA Damage Tolerance

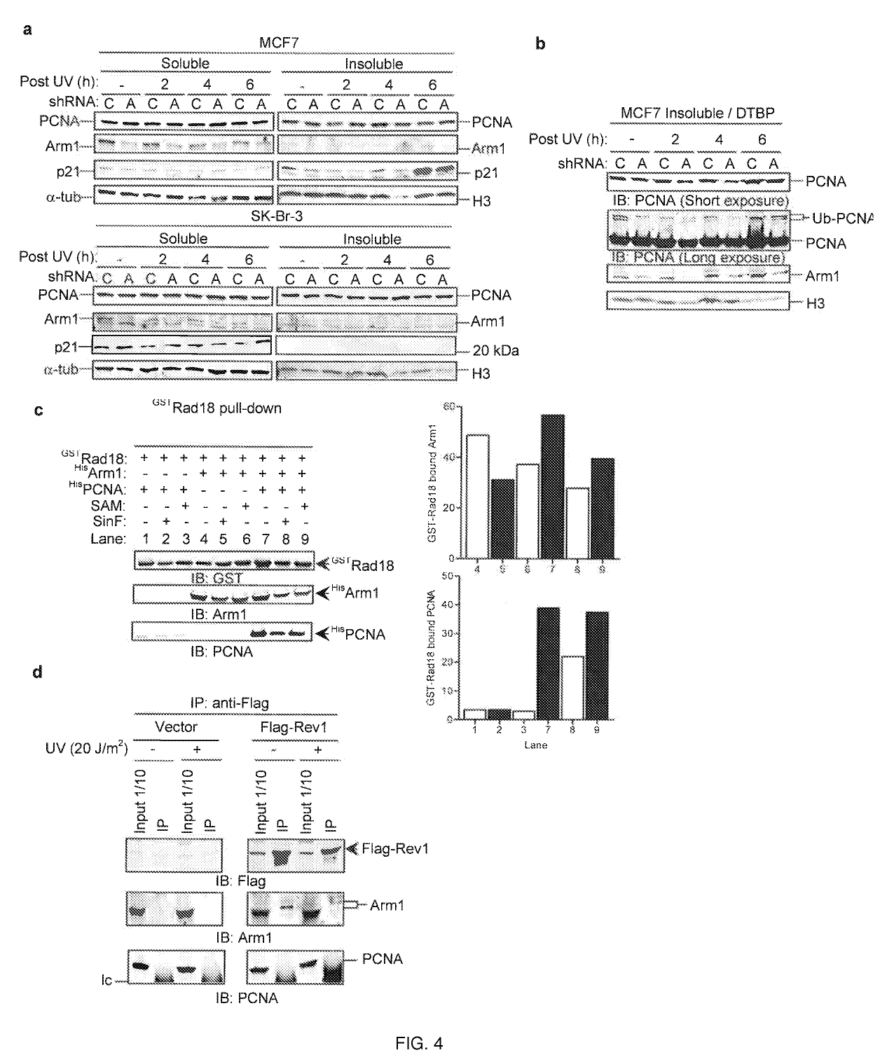

The results from this example are shown in FIG. 4. a, PCNA chromatin stability is un affected by Arm1. MCF7 and SK-Br-3 cell expressing shRNA were UV-irradiated (20 J/m²) for the indicated times and fractionated to Triton X-100 soluble and chromatin bound insoluble fractions. Fractions were separated by SDS-PAGE and immunoblotted for PCNA, Arm1, and p21. a-tubulin and histone H3 were used as loading controls. b, Arm1 is recruited to the chromatin and promotes PCNA ubiquitylation. MCF7 cells expressing Arm1 shRNA were UV-irradiated (20 J/m²) and proteins cross-linked with DTBP at the indicated times prior to Triton X-100 extraction. Proteins present in the insoluble fraction were analyzed by SDS-PAGE and immunoblotting for PCNA and Arm1. Histone H3 served as a loading control. c, Arm1-dependent methyltransferase activity promotes Rad18's interactions with Arm1 and PCNA. Purified recombinant PCNA was incubated in the absence and presence of purified recombinant Arm1 with and without SAM (10 mM) or sinefungin (20 mM) for 1 h at 37° C. prior to rocking with glutathione Sepharose bound GST-Rad18 for 15 m at 4° C. The GST-Rad18 beads were washed and analyzed by SDS-PAGE and immunoblotting. d, Arm1 interacts with Rev1. SK-Br-3 cells were transfected with control or Flag-Rev1 expression plasmids and incubated for 24 h. Cells were harvested 6 h after UV irradiation.

### Example 9

### Model for PCNA Methyl Esterification

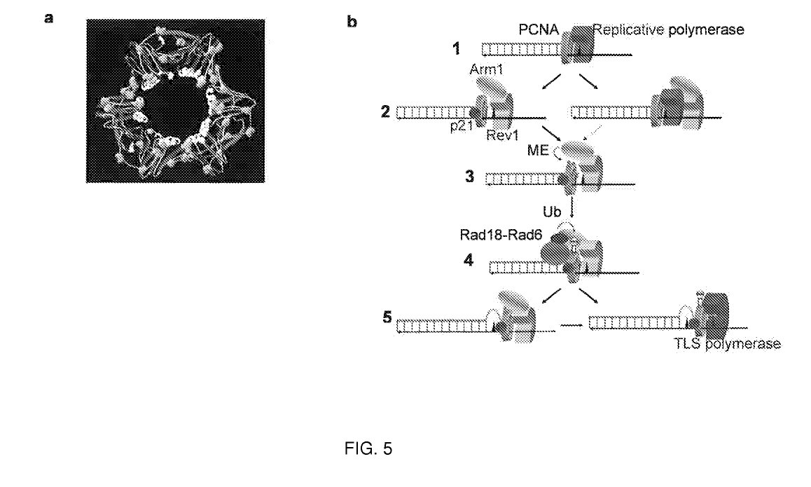

The results from this example are shown in FIG. 5. Methyl esterified PCNA residues identified by LC-MS/MS sequencing of the p21-PIP affinity purified isoforms (FIG. 3d, table I, and FIG. 10) are shown (orange) on the PCNA structure described in Gulbis et al.²¹. PCNA subunits are shown in blue, green and magenta, and the p21 PIP-box peptide is shown in red. DNA damage induces up-regulation of p21 and methyl esterification of PCNA. Knock-down of Arm1 expression promotes survival in p53 wild-type cells and cytotoxicity in p53-mutant cells.

### Example 10

### Identification of the C6orf211 Protein in the PCNA-Dependent Carboxyl Methyltransferase Active Fraction

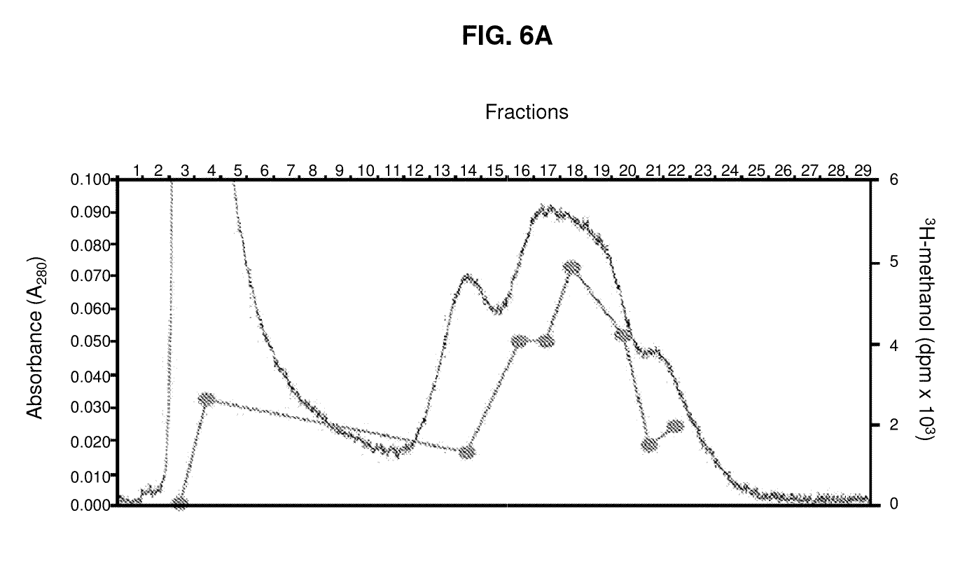

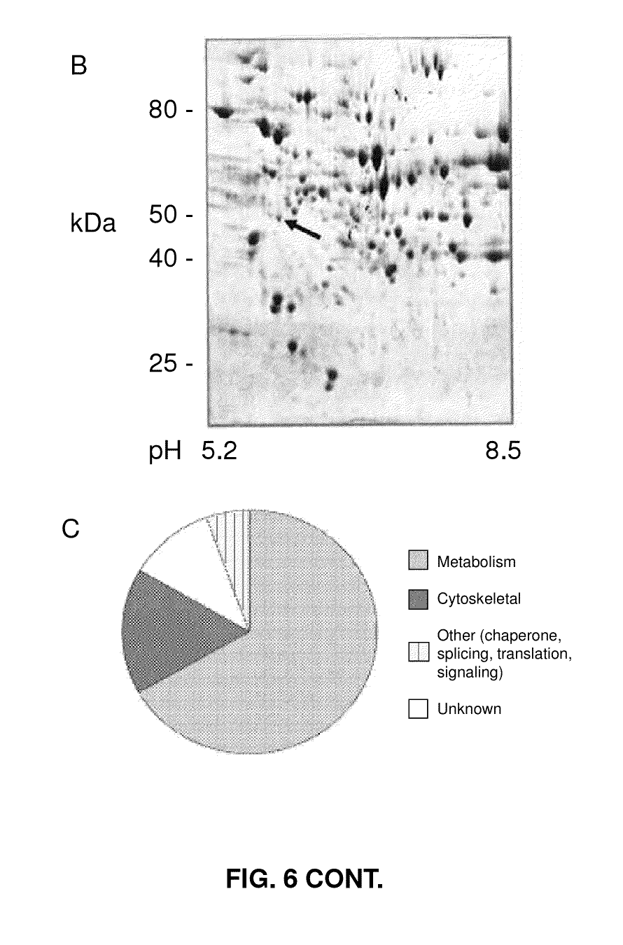

The results from this example are shown in FIG. 6. MDA MB468 cell extracts were subjected to 30% NH₄SO₄ precipitation prior to loading onto a phenyl Sepharose column elution with a linear gradient of NH₄SO₄ to 0% (a). Activity was further enriched by passage over a Superdex 5200 gel filtration column, and the active fractions resolved by 2D-PAGE and stained with colloidal Coomassie Blue (b). The position of the 50 kDa product of the uncharacterized gene C6orf211 is identified with an arrow. Proteins present in the enriched fractions were identified by mass spectrometry and grouped by cellular function (c).

### Example 11

### Sequence Alignments of CheR, C6orf211, and PIMT Proteins

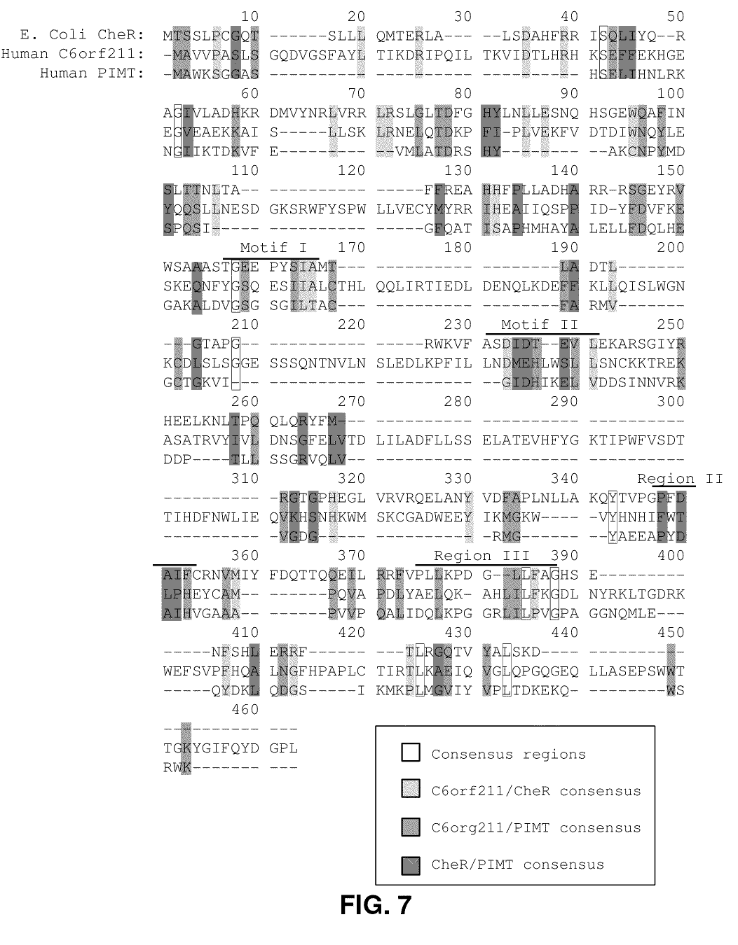

The results from this example are shown in FIG. 7. Full-length protein sequences of CheR, the C6orf211 gene product, and PIMT were aligned using MUSCLE⁴². Consensus sequences previously determined in CheR and PIMT are shown^{13,14}. Amino acids are colour coded green (polar), red (nonpolar, hydrophobic), pink (basic), and blue (acidic).

### Example 12

### Conservation of the C6orf211 Gene Product in Eukaryotes

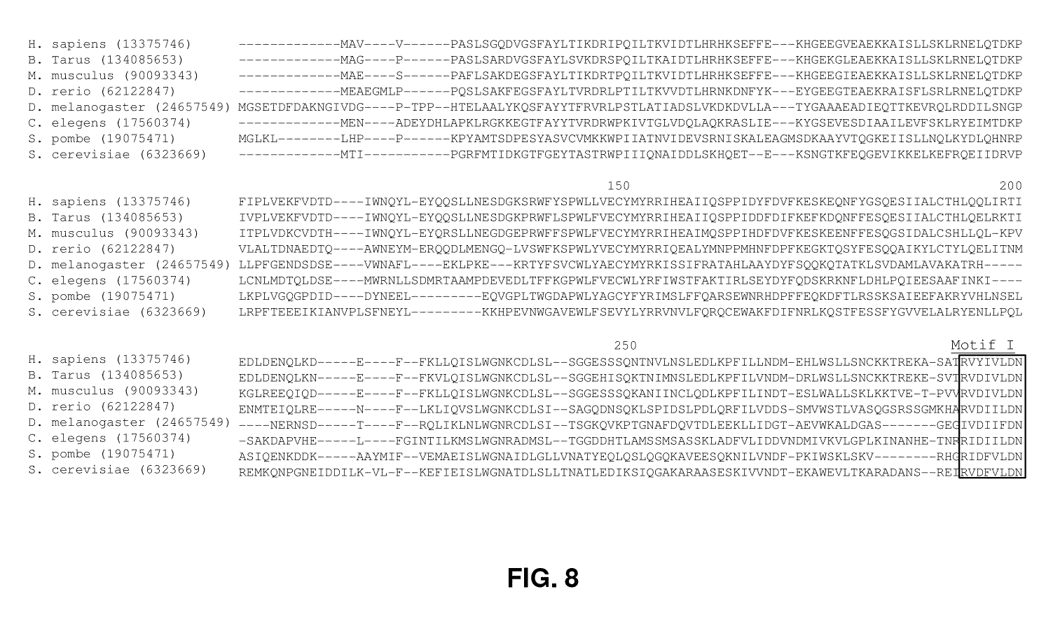

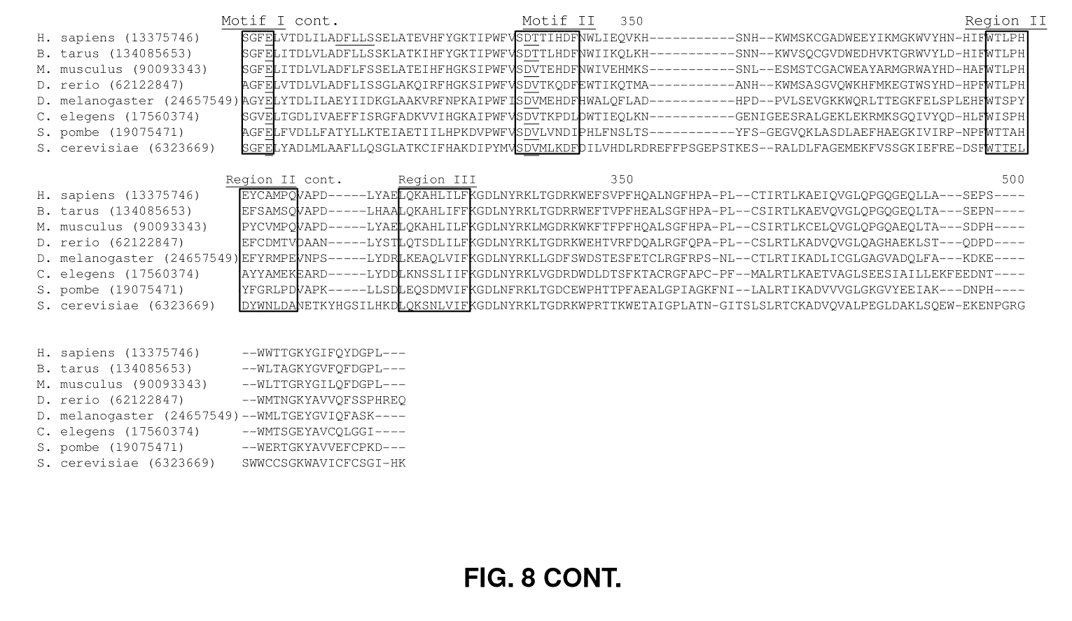

The results from this example are shown in FIG. 8. The C6orf211 proteins from eight eukaryotic organisms were aligned with KALIGN and motifs I and II and regions II and III identified. The motif I sequence shows high conservation among all organisms and its location in the primary sequence positions it in the b1/aA loop of the hypothetical SAM-MT fold shown in FIG. 1e. Conserved glycine residues of motif I are underlined. Identification of motif II was made using the hypothetical SAM-MT fold. The motif II sequence present in the b2/aB loop is also highly conserved among all species. Regions II and III, although less conserved, show significant conservation.

### Example 13

### Methyl Esterification of p21-PIP Affinity Purified PCNA Isoforms

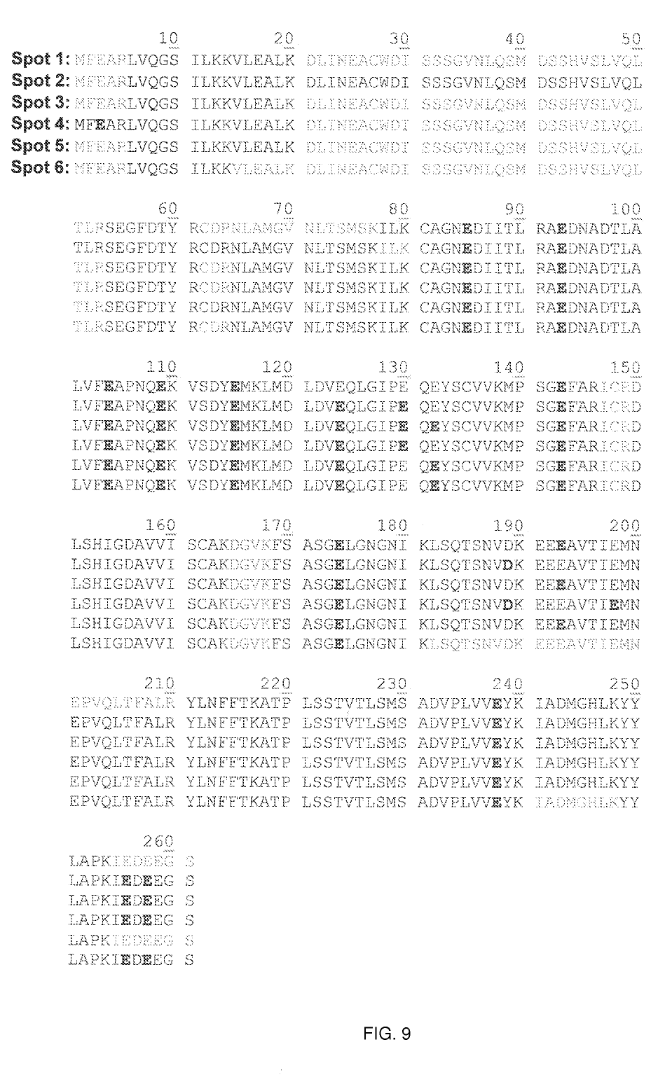

The results from this example are shown in FIG. 9. Affinity purified PCNA isoforms (FIG. 2) were separated and excised from 2D-PAGE gels and analyzed by LC-MS/MS. Positively identified peptide sequences are shown in black and unobserved sequences are shown in red. The locations of methyl esterified residues in the PCNA isoform spots are presented in bolded blue.

### Example 14

### Lentiviral shRNA Knock-Down of Arm Expression in MCF7 and SK-Br-3 Cells

The results from this example are shown in FIG. 10. a, MCF7 and SK-Br-3 cells were infected with lentiviral shRNA and bicistronic expression of TurboGFP confirmed shRNA expression. b, Arm1 mRNA expression is reduced in shRNA expressing cells. Relative Arm1 mRNA expression was determined by Q-PCR. Normalized expression levels are presented (±SD).

### Example 15

### Identification of Arm1

To determine if PCNA methyl esters were the result of a posttranslational mechanism we examined breast cancer cell extracts for it ability to methyl esterify PCNA (FIG. 1a). As a result we were able to detect a carboxyl methyltransferase activity in these extracts that was dependent on PCNA. To identify the enzyme responsible for methyl esterifying PCNA we subsequently enriched for PCNA-dependent carboxyl methyltransferase activity from extracts (FIG. 6 and table I). Using proteomics techniques then identified proteins comprising the active fractions, and the majority of proteins identified were of known function and were excluded from further consideration. From this approach we were able to rapidly narrow down methyltransferase candidates to an uncharacterized protein, the 50 kDa product of a hypothetical orf on chromosome 6 (C6orf211)¹². This hypothetical protein was further assessed for methyltransferase potential.

As an initial step we aligned the C6orf211 protein with the bacterial glutamyl methyltransferase CheR and the human isoaspartate methyltransferase PIMT (FIG. 7). Like many of the SAM-dependent methyltransferases (SAM-MT), the C6orf211 protein showed limited sequence conservation; however, the SAM-MTs share a common structure called the SAM-MT fold^{13,14} and we searched the protein for this fold. By the predicting C6orf211 protein's secondary structures we identified several structures that could assemble into a SAM-MT fold (FIG. 1e) in a pattern similar to CheR (FIG. 1d). Within the SAM-MT fold is the SAM binding pocket that possesses two highly conserved sequence motifs. Alignment of the CheR motifs with full-length C6orf211 protein identified similar sequences (FIG. 1b) that showed significant evolutionary conservation (FIG. 8). Furthermore motifs I and II were positioned in the β1/αA and β2/αB loops of our hypothetical SAM-MT fold (FIG. 1e). In addition to motifs I and II, two conserved regions (II and III) have been identified in CheR and PIMT¹⁴. Alignments of the CheR regions with the C6orf211 protein identified analogous sequences with significant evolutionary conservation (FIG. 1b and FIG. 8), which, with motifs I and II, were identified in similar positions in the C6orf211 protein as CheR (FIG. 1c). These results strongly suggested that this uncharacterized protein was a methyltransferase, and expression and purification confirmed that it possessed a PCNA-dependent carboxyl methyltransferase activity (FIG. 1f & 1g). Interestingly, in addition to methyl esterifying PCNA, the C6orf211 protein or Arm1 appeared to modify itself suggesting that it may be self-regulated.

### Example 16

### DNA Damage Promotes PCNA Methyl Esterification

Identification of the C6orf211 gene product as Arm1, a PCNA-dependent carboxyl methyltransferase, hinted at a novel mechanism occurring in eukaryotic cells. However, the biological significance of methyl esterification was uncertain. Therefore, we used 2D-PAGE to search for PCNA methyl esterification in MCF7 breast cancer cells following DNA damage, which would be identified by a basic shift in isoelectric point (pI). In untreated cells PCNA displays a pI at or near its theoretical value of 4.5^{10,15}. But following treatment with the DNA damaging agent doxorubicin (Dox), a basic PCNA isoform (pI ˜5.6) was observed (FIG. 2a). This PCNA isoform was consistent with methyl esterification of 15+ acidic residues, and assaying the extracts for Arm1 activity showed a wave of PCNA methyl esterification that increased two hours after Dox exposure, shifted to reduced activity by 4 h, and stabilized after 6 h (FIG. 2b). This suggested that PCNA methyl esterification builds and peaks by 4 h in MCF7 cells, and by that time PCNA methyl esterification is inhibited and/or PCNA methyl esterase activity predominates over methyltransferase activity. Interestingly, increased PCNA-dependent carboxyl methyltransferase activity and the appearance of the basic PCNA isoform correlated with a >27-fold increased expression of p21^{WAF1/CIP1} in MCF7 cells (FIG. 2c).

Since the initial observations describing p21's interaction with PCNA and inhibition of DNA replication in response to DNA damage¹⁶, the function of the p21-PCNA interaction in the DNA damage response has remained poorly understood. In addition to DNA replication, PCNA is required for DNA repair, and p21 or the PCNA interacting peptide (PIP) of p21 have been shown to disrupt mismatch¹⁷, base excision¹⁸, and nucleotide excision repair¹⁹. Despite this inhibition of DNA repair, however, p21^{-/-} cells display a repair defect²⁰. It was therefore possible that PCNA methyl esterification could further our understanding of p21 in the DNA damage response, so we investigated PCNA methyl esterification and the p21-PCNA interaction (FIG. 2d). To our surprise, the p21 interaction had a direct effect on PCNA methyl esterification in untreated cell extracts and by pulling down PCNA from breast cancer cell extracts with a GST-p21 fusion we observed basic shifted PCNA isoform induced by Dox exposure. This isoform was not evident in the input extracts so it was unlikely that its appearance was the result of enrichment. Instead, these results indicated that p21 regulated PCNA methyl esterification, and that the PIP domain of p21 could produce this basic shifted isoform. Using the p21(PIP) peptide we then affinity purified PCNA (FIG. 2e) to determine if methyl esterification was indeed promoting its basic pI shift. By sequencing the affinity purified PCNA isoforms we were able to identify a consistent trend of increasing methyl esterification on the basic-shifted PCNA isoforms (table I). The positions of the methyl esters on these isoforms were also highly conserved, and appeared nearly exclusive on glutamate residues (FIG. 10). Several methyl esters were also identified at previously unrecognized positions (E124, E130, E193 and E198), and PCNA's C-terminus was found exclusively di-methyl esterified. This was an intriguing result considering our previous observations of mono-methyl esters on two separate residues of the acidic PCNA isoform¹⁰ and its involvement in the p21 interaction²¹. Together these data strongly supported regulation of PCNA methyl esterification by p21, and to further explore this mechanism we knocked down Arm1 expression in p53 wild-type and p53-muant cells and examined their abilities to respond to DNA damage.

### Example 17

### Arm1-Dependent PCNA Methyl Esterification is Linked to DNA Repair

Although the previous results support PCNA methyl esterification in the DNA damage response, the role of Arm1 in this response was still unclear. Therefore, we knocked-down Arm1 expression in MCF7(p53 wild-type) and SK-Br-3 (p53-mutant) breast cancer cells (FIG. 10). We then damaged the DNA of these cells with Dox and UV and examined PCNA mobility by 2D-PAGE (FIG. 3a). Unexpectedly, methyl esterified PCNA isoforms were evident in untreated MCF7 following Arm1 knockdown. Although the appearance of the basic isoform in the undamaged extracts could be attributed to residual Arm1 expression, it also indicated that Arm1 knockdown dysregulated PCNA modification, and in yeast Arm1 mutant cells. Additionally, posttranslational state of PCNA in Dox treated Arm1 knockdown MCF7 cells appeared drastically different to the control cells. However, the clearest results were observed following UV irradiation. A PCNA isoform indicative of methyl esterification was observed in UV treated shRNA control cells, but not in the Arm1 shRNA expressing cells (FIG. 3a) indicating that methyl esterification is also functional in response to UV. The drastic differences in PCNA's posttranslational states following Dox and UV exposures could be explained by the nature of the DNA damage generated by these agents. Although Dox generates DNA double strand breaks through topoisomerase inhibition as well as inhibition of transcription and DNA replication, it is also implicated in alkylation, cross-linking, and free-radical DNA damage²². Compared to UV damage, which predominantly generates pyrimidine dimers, Dox-generated DNA damage would necessitate multiple modes of DNA repair and PCNA's interactions with several repair factors. The appearance of numerous PCNA isoforms in the Dox treated Arm1 shRNA expressing cells could therefore be indicative of a level of control over protein-protein interactions that promote PCNA posttranslational modifications in response to numerous types of DNA damage.

In addition to p53 wild-type MCF7 cells we also examined p53-mutant SK-Br-3 cells that are unable to induce p21 expression following DNA damage. Interestingly, methyl esterified PCNA isoforms were also observed in the control cells following Dox and UV exposures and were significantly reduced in the Arm1 knockdown cells (FIG. 3a). Like MCF7 cells, loss of PCNA methyl esterification was most apparent in the SK-Br-3 Arm1 knockdown cells following UV-irradiation suggesting that PCNA methyl esterification does occur in the absence of p21 up-regulation. The role of p21 in response to UV-induced DNA damage is controversial partially due to its ability to disrupt PCNA's interaction of with nucleotide excision repair endonuclease XPG¹⁹ and p21 degradation has been shown to promote UV repair²³. We therefore examined p21 expression in these cell lines following UV-irradiation (FIG. 3c). Although p21 up-regulation was not observed in SK-Br-3 cells, we did observe the accumulation of chromatin bound p21 in MCF7 cells. This suggested that p21 exerts its affects on chromatin bound PCNA in MCF7 cells in response to UV damage. PCNA chromatin stability was fairly constant in the Arm1 knockdown cells compared to the controls in response to UV with slightly higher levels of chromatin bound PCNA were observed in the Arm1 knockdown cells 2 and 6 h following UV (FIG. 3c). Arm1 levels were very low in the chromatin bound fractions suggestive of EGFR-dependent phosphorylation has been shown to protect PCNA from ubiquitin-dependent degradation and promoting chromatin stability²⁴. It was therefore possible that ubiquitin-dependent PCNA degradation and removal from the chromatin.suggesting that Arm1 has a minimal although slightly increased levels of PCNA are slightly observed of The present on the chromatinp21 on the Additionally, contradictory results on p21's role in PCNA ubiquitylation and translesion DNA synthesis (TLS) have also been reported^{25,26} is also unclear and contradictory results. Arm1 could therefore be a missing factor that may help explain some of these contradictory findings when DNA damage tolerance has been observed. This suggested that there are p21-independent roles for Arm1 in the DNA damage response as well. The DNA damage-induced PCNA isoform appeared nearly unaltered in both Arm1 shRNA expressing cell lines in response to the alkylating agent MMS when compared to the controls suggesting a limited role for Arm1 in response to this type of DNA damage. However, a slower migrating potentially ubiquitylated or SUMOylated PCNA species was observed in the control and not Arm1 shRNA expressing MCF7 cells (FIG. 4a) suggesting that Arm1 could have a role in post-replication DNA repair. The absence of this slower migrating PCNA species in MMS treated SK-Br-3 cells was consistent with previous observations that p53 and p21 promoted UV-induced PCNA ubiquitylation, and that expression of the p21-PIP box was sufficient to suppress efficiency and increased fidelity of translesion DNA synthesis activity in cells²⁶. In contrast, expression of a non-degradable p21 mutant was shown to inhibit PCNA ubiquitylation following UV damage²⁵. Regardless of this discrepancy, p21 does appear to affect PCNA ubiquitylation, which further implicates Arm1 in post-replication DNA repair.

In addition to PCNA modification in knock-down cells, we examined cell survival following DNA damage (FIG. 4b). Interestingly, cell survival in response to DOX, MMS, and UV were significantly different in the Arm knock-down cells compared to control cells. Knock-down of Arm1 in p53 wild-type MCF7 cells led to significantly enhanced survival in response to DOX, MMS, and UV. In MCF7 cells with knocked-down Arm1 expression 47% of cells survived 0.5 mM DOX compared to 15% survival in the control cells (FIG. 4b). Similar results were consistently observed in response to UV with 39% of the Arm1 knock-down cells surviving 25 J/m² compared to 19% in the control. And although not as dramatic, enhanced survival was also observed in response to MMS. In stark contrast to MCF7 cells, knock-down of Arm1 expression in p53-mutant SK-Br-3 cells significantly reduced survival to only 3% in response to 0.5 mM DOX compared to 18% of the control cells. These results were also consistent in UV and MMS treatments with 4% and 48% of Arm1 knock-down cells surviving 0.0025% MMS and 25 J/m² UV compared to 12% and 82% in the control cells, respectively. These results strongly supported Arm1's function in DNA repair, and suggested that this novel signalling mechanism plays an important role in the cell's ability to properly respond to and repair DNA damage. Additionally, because ˜50% of tumours harbour p53 mutations and knock-down of Arm1 expression in p53-mutant breast tumours appeared to sensitize the cells to DNA damage, inhibition of Arm1 may ultimately allow us to more effectively target tumour cells.

### DISCUSSION

We describe a novel eukaryotic protein carboxyl methyltransferase, Arm1, which specifically targets glutamic and aspartic acid residues in PCNA. We also present evidence that methyl esterification of PCNA is stimulated following exposure of cells to genotoxic stress, which is mediated, at least in part, through p21 binding. As early as 1979, the methyl esterification of glutamic acid residues in the eukaryotic proteins was reported by what was, at that time, known as protein carboxyl O-methyltransferase²⁷. Subsequently, protein carboxyl O-methyltransferase's specificity for iso-aspartate residues and ability to facilitate protein repair led to its reassignment as protein isoaspartate methyltransferase (PIMT)²⁸. Since that time, investigations into glutamyl methyl esterification of eukaryotic proteins have been essentially nonexistent. With the advent of proteomics and advances in modern protein mass spectrometry, the unambiguous detection of these structures on eukaryotic proteins has become possible. And since our initial observations¹⁰, at least two independent laboratories have described these structures on aspartic and glutamic acid residues in eukaryotic proteins^{29,30}.

How methyl esterification affects PCNA's structure remains to be elucidated; but, in prokaryotic cells, chemotaxis receptor methyl esterification changes it conformations controlling protein-protein interactions effecting the cell's ability to adapt to stimuli³¹. Likewise, Arm1-dependent methyl esterification of PCNA may regulate its protein-protein interactions ultimately allowing the cell to adapt to genotoxic stress. Examination of the positions of methyl esterified residues on the PCNA crystal structure²¹ (FIG. 5) identified a concentration of these structures in the subunit interfaces indicating potential effects on trimer assembly. This is supported by observations that a glutamic acid to glycine (E113G) mutation in the subunit interface of yeast PCNA significantly affected the molecule's ability to form trimers³². Also consistent with this is the reported correlation between UV-dependent ubiquitylation and degradation of p21 and the accumulation of chromatin bound PCNA²³. This further suggests that PCNA methyl esterification following p21 binding may promote PCNA disassembly from chromatin. Regardless of Arm1's mechanism, its presence appears to be required for appropriate response to DNA damage, and the data reported here may help explain some contradictory observations on p21's tumour suppressor and oncogenes functions^{33,34,35}.

In addition to PCNA, Arm1 likely has multiple other targets and it is difficult to speculate as to whether the survival differences observed in the Arm1 knockdown cells were mediated solely through PCNA methyl esterification. However, an interaction of PCNA with ING1 was previously shown to promote UV-induced apoptosis and prevention of this interaction through either over-expression of p21 or mutation to ING1's PCNA interacting PIP-box prevented UV-induced apoptosis³⁶. It is therefore attractive to postulate that Arm1 could regulate PCNA's interactions with, among other factors, ING. And loss of Arm1's ability to regulate PCNA's interactions may prevent the cell from effectively responding to DNA damage. Further investigations are required to determine Arm′1 exact role(s) in response to genotoxic stress, but from these results it is clear that methyl esterification of acidic protein residues is a real posttranslational mechanism that alters protein structure and function in eukaryotes.

### REFERENCES

The following references reflect the level of knowledge in the field and are hereby incorporated by reference in their entirety. Any conflict between the teachings of these references and this specification shall be resolved in favor of the latter.
¹ Moldovan, G. L., Pfander, B. & Jentsch, S. PCNA, the maestro of the replication fork. Cell 129, 665-679, doi:S0092-8674(07)00594-6 [pii] 10.1016/j.cell.2007.05.003 (2007).
² Andersen, P. L., Xu, F. & Xiao, W. Eukaryotic DNA damage tolerance and translesion synthesis through covalent modifications of PCNA. Cell Res 18, 162-173, doi:cr2007114 [pii] 10.1038/cr.2007.114 (2008).
³ Ulrich, H. D. Conservation of DNA damage tolerance pathways from yeast to humans. Biochem Soc Trans 35, 1334-1337, doi:BST0351334 [pii] 10.1042/BST0351334 (2007).
⁴ Ulrich, H. D. Regulating post-translational modifications of the eukaryotic replication clamp PCNA. DNA Repair (Amst) 8, 461-469, doi:S1568-7864(09)00011-1 [pii] 10.1016/j.dnarep.2009.01.006 (2009).
⁵ Ulrich, H. D. & Walden, H. Ubiquitin signalling in DNA replication and repair. Nat Rev Mol Cell Biol 11, 479-489, doi:nrm2921 [pii] 10.1038/nrm2921.
⁶ Hoege, C., Pfander, B., Moldovan, G. L., Pyrowolakis, G. & Jentsch, S. RAD6-dependent DNA repair is linked to modification of PCNA by ubiquitin and SUMO. Nature 419, 135-141, doi:10.1038/nature00991 nature00991 [pii] (2002).
⁷ Stelter, P. & Ulrich, H. D. Control of spontaneous and damage-induced mutagenesis by SUMO and ubiquitin conjugation. Nature 425, 188-191, doi:10.1038/nature01965 nature01965 [pii] (2003).
⁸ Kannouche, P. L., Wing, J. & Lehmann, A. R. Interaction of human DNA polymerase eta with monoubiquitinated PCNA: a possible mechanism for the polymerase switch in response to DNA damage. Mol Cell 14, 491-500, doi:S109727650400259X [pii] (2004).
⁹ Branzei, D., Vanoli, F. & Foiani, M. SUMOylation regulates Rad18-mediated template switch. Nature 456, 915-920, doi:nature07587 [pii] 10.1038/nature07587 (2008).
¹⁰ Hoelz, D. J. et al. The discovery of labile methyl esters on proliferating cell nuclear antigen by MS/MS. Proteomics 6, 4808-4816, doi:10.1002/pmic.200600142 (2006).
¹¹ Grillo, M. A. & Colombatto, S. S-adenosylmethionine and protein methylation. Amino Acids 28, 357-362, doi:10.1007/s00726-005-0197-6 (2005).
¹² Chromosome 6 open reading frame 211 [Homo sapiens]: Protein accession numbers: RefSeq: NM_{—}024573.1; GenBank: AAH11348; UniprotKB: Q9H993.
¹³ Martin, J. L. & McMillan, F. M. SAM (dependent) I AM: the S-adenosylmethionine-dependent methyltransferase fold. Curr Opin Struct Biol 12, 783-793, doi:S0959440X02003913 [pii] (2002).
¹⁴ Ingrosso, D., Fowler, A. V., Bleibaum, J. & Clarke, S. Sequence of the D-aspartyl/L-isoaspartyl protein methyltransferase from human erythrocytes. Common sequence motifs for protein, DNA, RNA, and small molecule S-adenosylmethionine-dependent methyltransferases. J Biol Chem 264, 20131-20139 (1989).
¹⁵ Malkas, L. H. et al. A cancer-associated PCNA expressed in breast cancer has implications as a potential biomarker. Proc Natl Acad Sci USA 103, 19472-19477, doi:0604614103 [pii] 10.1073/pnas.0604614103 (2006).
¹⁶ Waga, S., Hannon, G. J., Beach, D. & Stillman, B. The p21 inhibitor of cyclin-dependent kinases controls DNA replication by interaction with PCNA. Nature 369, 574-578, doi:10.1038/369574a0 (1994).
¹⁷ Umar, A. et al. Requirement for PCNA in DNA mismatch repair at a step preceding DNA resynthesis. Cell 87, 65-73, doi:S0092-8674(00)81323-9 [pii] (1996).
¹⁸ Tom, S., Ranalli, T. A., Podust, V. N. & Bambara, R. A. Regulatory roles of p21 and apurinic/apyrimidinic endonuclease 1 in base excision repair. J Biol Chem 276, 48781-48789, doi:10.1074/jbc.M109626200 M109626200 [pii] (2001).
¹⁹ Gary, R., Ludwig, D. L., Cornelius, H. L., MacInnes, M. A. & Park, M. S. The DNA repair endonuclease XPG binds to proliferating cell nuclear antigen (PCNA) and shares sequence elements with the PCNA-binding regions of FEN-1 and cyclin-dependent kinase inhibitor p21. J Biol Chem 272, 24522-24529 (1997).
²⁰ McDonald, E. R., 3rd, Wu, G. S., Waldman, T. & El-Deiry, W. S. Repair Defect in p21 WAF1/CIP1−/− human cancer cells. Cancer Res 56, 2250-2255 (1996).
²¹ Gulbis, J. M., Kelman, Z., Hurwitz, J., O'Donnell, M. & Kuriyan, J. Structure of the C-terminal region of p21(WAF1/CIP1) complexed with human PCNA. Cell 87, 297-306, doi:S0092-8674(00)81347-1 [pii] (1996).
²² Gewirtz, D. A. A critical evaluation of the mechanisms of action proposed for the antitumor effects of the anthracycline antibiotics adriamycin and daunorubicin. Biochem Pharmacol 57, 727-741, doi:S0006295298003074 [pii] (1999).
²³ Bendjennat, M. et al. UV irradiation triggers ubiquitin-dependent degradation of p21(WAF1) to promote DNA repair. Cell 114, 599-610, doi:S0092867403006445 [pii] (2003).
²⁴ Wang, S. C. et al. Tyrosine phosphorylation controls PCNA function through protein stability. Nat Cell Biol 8, 1359-1368, doi:ncb1501 [pii] 10.1038/ncb1501 (2006).
²⁵ Soria, G., Podhajcer, O., Prives, C. & Gottifredi, V. P21Cip1/WAF1 downregulation is required for efficient PCNA ubiquitination after UV irradiation. Oncogene 25, 2829-2838, doi:1209315 [pii] 10.1038/sj.onc.1209315 (2006).
²⁶ Avkin, S. et al. p53 and p21 regulate error-prone DNA repair to yield a lower mutation load. Mol Cell 22, 407-413, doi:S1097-2765(06)00188-2 [pii] 10.1016/j.molcel.2006.03.022 (2006).
²⁷ Kim, S. & Li, C. H. Enzymatic methyl esterification of specific glutamyl residue in corticotropin. Proc Natl Acad Sci USA 76, 4255-4257 (1979).
²⁸ Johnson, B. A., Murray, E. D., Jr., Clarke, S., Glass, D. B. & Aswad, D. W. Protein carboxyl methyltransferase facilitates conversion of atypical L-isoaspartyl peptides to normal L-aspartyl peptides. J Biol Chem 262, 5622-5629 (1987).
²⁹ Sprung, R. et al. Identification and validation of eukaryotic aspartate and glutamate methylation in proteins. J Proteome Res 7, 1001-1006, doi:10.1021/pr0705338 (2008).
³⁰ Lesner, A. et al. Identification of X-DING-CD4, a new member of human DING protein family that is secreted by HIV-1 resistant CD4(+) T cells and has anti-viral activity. Biochem Biophys Res Commun 389, 284-289, doi:S0006-291X(09)01730-6 [pii] 10.1016/j.bbrc.2009.08.140 (2009).
³¹ Jurica, M. S. & Stoddard, B. L. Mind your B's and R's: bacterial chemotaxis, signal transduction and protein recognition. Structure 6, 809-813 (1998).
³² Freudenthal, B. D., Gakhar, L., Ramaswamy, S. & Washington, M. T. A charged residue at the subunit interface of PCNA promotes trimer formation by destabilizing alternate subunit interactions. Acta Crystallogr D Biol Crystallogr 65, 560-566, doi:S0907444909011329 [pii] 10.1107/S0907444909011329 (2009).
³³ Gartel, A. L. p21(WAF1/CIP1) and cancer: a shifting paradigm? Biofactors 35, 161-164, doi:10.1002/biof.26 (2009).
³⁴ Prives, C. & Gottifredi, V. The p21 and PCNA partnership: a new twist for an old plot. Cell Cycle 7, 3840-3846, doi:7243 [pii] (2008).
³⁵ Abbas, T. & Dutta, A. p21 in cancer: intricate networks and multiple activities. Nat Rev Cancer 9, 400-414, doi:nrc2657 [pii] 10.1038/nrc2657 (2009).
³⁶ Scott, M. et al. UV-induced binding of ING1 to PCNA regulates the induction of apoptosis. J Cell Sci 114, 3455-3462 (2001).
³⁷ Murray, E. D., Jr. & Clarke, S. Synthetic peptide substrates for the erythrocyte protein carboxyl methyltransferase. Detection of a new site of methylation at isomerized L-aspartyl residues. J Biol Chem 259, 10722-10732 (1984).
³⁸ Podust, V. N., Podust, L. M., Goubin, F., Ducommun, B. & Hubscher, U. Mechanism of inhibition of proliferating cell nuclear antigen-dependent DNA synthesis by the cyclin-dependent kinase inhibitor p21. Biochemistry 34, 8869-8875 (1995).
³⁹ Lassmann, T. & Sonnhammer, E. L. Kalign, Kalignvu and Mumsa: web servers for multiple sequence alignment. Nucleic Acids Res 34, W596-599, doi:34/suppl_{—}2/W596 [pii] 10.1093/nar/gkl191 (2006).
⁴⁰ Hernandez, D., Gras, R. & Appel, R. Neighborhood functions and hill-climbing strategies dedicated to the generalized ungapped local multiple alignment. European Journal of Operational Research 185, 1276-1284 (2008).
⁴¹ Cole, C., Barber, J. D. & Barton, G. J. The Jpred 3 secondary structure prediction server. Nucl. Acids Res. 36, W197-201, doi:10.1093/nar/gkn238 (2008).
⁴² Edgar, R. C. MUSCLE: multiple sequence alignment with high accuracy and high throughput. Nucleic Acids Res 32, 1792-1797, doi:10.1093/nar/gkh340 32/5/1792 [pii] (2004).

1. A method for treating a mammal with cancer, the method comprising administering to the mammal a pharmaceutical formulation comprising an inhibitor of acidic residue methyltransferase (Arm1) in combination with a DNA damaging agent, wherein the inhibitor of Arm1 enhances the efficacy and selectivity of cancer cell killing by the DNA damaging agent.

2. (canceled)

3. A method for enhancing efficacy and selectivity of cancer cell killing by a DNA damaging agent, comprising contacting a tumor cell with an inhibitor of Arm1 in combination with a DNA damaging agent, wherein the inhibitor of Arm1 enhances the efficacy and selectivity of cancer cell killing by the DNA damaging agent.

4. The method of claim 3, wherein the cancer cell is in the body of a mammal.

5. The method of claim 3, wherein the DNA damaging agent is selected from the group consisting of doxorubicin, 6-mercaptopurine, gemcitabine, cyclophosphamide, melphalan, busulfan, chlorambucil, mitomycin, cisplatin, bleomycin, dectinomycin, irinotecan, and mitoxantrane.

6. The method of claim 1, wherein the DNA damaging agent is selected from the group consisting of doxorubicin, 6-mercaptopurine, gemcitabine, cyclophosphamide, melphalan, busulfan, chlorambucil, mitomycin, cisplatin, bleomycin, dectinomycin, irinotecan, and mitoxantrane.


SEQUENCE LISTING (25 sequences)

SEQ ID NO: 1 - protein, 13 aa, Artificial Sequence
  Synthetic - Motif I CheR (a.a. 117-129)
YRVWSAAASTGEE

SEQ ID NO: 2 - protein, 13 aa, Artificial Sequence
  Synthetic - Motif I Arm1 (a.a. 245-257)
TRVYIVLDNSGFE

SEQ ID NO: 3 - protein, 9 aa, Artificial Sequence
  Synthetic - Motif II CheR (a.a. 152-160)
ASDIDTEVL

SEQ ID NO: 4 - protein, 9 aa, Artificial Sequence
  Synthetic - Motif II Arm1(a.a. 289-297)
VSDTTIHDF

SEQ ID NO: 5 - protein, 12 aa, Artificial Sequence
  Synthetic - Consensus region II CheR (a.a. 218-229)
YTVPGPFDAIFC

SEQ ID NO: 6 - protein, 12 aa, Artificial Sequence
  Synthetic - Consensus region II Arm1 (a.a. 336-347)
WTLPHEYCAMPQ

SEQ ID NO: 7 - protein, 9 aa, Artificial Sequence
  Synthetic - Consensus region III CheR (a.a. 251-259)
LLKPDGLLF

SEQ ID NO: 8 - protein, 9 aa, Artificial Sequence
  Synthetic - Consensus region III Arm1 (a.a. 356-364)
LQKAHLILF

SEQ ID NO: 9 - protein, 286 aa, E.coli
  misc_feature (1)..(286): E.coli CheR
MTSSLPCGQTSLLLQMTERLALSDAHFRRISQLIYQRAGIVLADHKRDMVYNRLVRRLRSLGLTDFGHYLNLLESNQHSGEWQAFINSLTTNLTAFFREAHHFPLLADHARRRSGEYRVWSAAASTGEEPYSIAMTLADTLGTAPGRWKVFASDIDTEVLEKARSGIYRHEELKNLTPQQLQRYFMRGTGPHEGLVRVRQELANYVDFAPLNLLAKQYTVPGPFDAIFCRNVMIYFDQTTQQEILRRFVPLLKPDGLLFAGHSENFSHLERRFTLRGQTVYALSKD

SEQ ID NO: 10 - protein, 441 aa, Homo sapiens
  misc_feature (1)..(441): Human C6orf211
MAVVPASLSGQDVGSFAYLTIKDRIPQILTKVIDTLHRHKSEFFEKHGEEGVEAEKKAISLLSKLRNELQTDKPFIPLVEKFVDTDIWNQYLEYQQSLLNESDGKSRWFYSPWLLVECYMYRRIHEAIIQSPPIDYFDVFKESKEQNFYGSQESIIALCTHLQQLIRTIEDLDENQLKDEFFKLLQISLWGNKCDLSLSGGESSSQNTNVLNSLEDLKPFILLNDMEHLWSLLSNCKKTREKASATRVYIVLDNSGFELVTDLILADFLLSSELATEVHFYGKTIPWFVSDTTIHDFNWLIEQVKHSNHKWMSKCGADWEEYIKMGKWVYHNHIFWTLPHEYCAMPQVAPDLYAELQKAHLILFKGDLNYRKLTGDRKWEFSVPFHQALNGFHPAPLCTIRTLKAEIQVGLQPGQGEQLLASEPSWWTTGKYGIFQYDGPL

SEQ ID NO: 11 - protein, 227 aa, Homo sapiens [sequence omitted]

SEQ ID NO: 12 - protein, 441 aa, Homo sapiens; identical to SEQ ID NO: 10
  misc_feature (1)..(441): H. sapiens (13375746)

SEQ ID NO: 13 - protein, 441 aa, B. taurus
  misc_feature (1)..(441): B. taurus (134085653)
MAGPPASLSARDVGSFAYLSVKDRSPQILTKAIDTLHRHKSEFFEKHGEKGLEAEKKAISLLSKLRNELQTDKPIVPLVEKFVDTDIWNQYLEYQQSLLNESDGKPRWFLSPWLFVECYMYRRIHEAIIQSPPIDDFDIFKEFKDQNFFESQESIIALCTHLQELRKTIEDLDENQLKNEFFKVLQISLWGNKCDLSLSGGEHISQKTNIMNSLEDLKPFILVNDMDRLWSLLSNCKKTREKESVTRVDIVLDNSGFELITDLVLADFLLSSKLATKIHFYGKTIPWFVSDTTLHDFNWIIKQLKHSNNKWVSQCGVDWEDHVKTGRWVYLDHIFWTLPHEFSAMSQVAPDLHAALQKAHLIFFKGDLNYRKLTGDRRWEFTVPFHEALSGFHPAPLCSIRTLKAEVQVGLQPGQGEQLTASEPNWLTAGKYGVFQFDGPL

SEQ ID NO: 14 - protein, 439 aa, M. musculus
  misc_feature (1)..(439): M. musculus (90093343)
MAESPAFLSAKDEGSFAYLTIKDRTPQILTKVIDTLHRHKSEFFEKHGEEGIEAEKKAISLLSKLRNELQTDKPITPLVDKCVDTHIWNQYLEYQRSLLNEGDGEPRWFFSPWLFVECYMYRRIHEAIMQSPPIHDFDVFKESKEENFFESQGSIDALCSHLLQLKPVKGLREEQIQDEFFKLLQISLWGNKCDLSLSGGESSSQKANIINCLQDLKPFILINDTESLWALLSKLKKTVETPVVRVDIVLDNSGFELITDLVLADFLFSSELATEIHFHGKSIPWFVSDVTEHDFNWIVEHMKSSNLESMSTCGACWEAYARMGRWAYHDHAFWTLPHPYCVMPQVAPDLYAELQKAHLILFKGDLNYRKLMGDRKWKFTFPFHQALSGFHPAPLCSIRTLKCELQVGLQPGQAEQLTASDPHWLTTGRYGILQFDGPL

SEQ ID NO: 15 - protein, 448 aa, D. rerio
  misc_feature (1)..(448): D. rerio (62122847)
MEAEGMLPPQSLSAKFEGSFAYLTVRDRLPTILTKVVDTLHRNKDNFYKEYGEEGTEAEKRAISFLSRLRNELQTDKPVLALTDNAEDTQAWNEYMERQQDLMENGQLVSWFKSPWLYVECYMYRRIQEALYMNPPMHNFDPFKEGKTQSYFESQQAIKYLCTYLQELITNMENMTEIQLRENFLKLIQVSLWGNKCDLSISAGQDNSQKLSPIDSLPDLQRFILVDDSSMVWSTLVASQGSRSSGMKHARVDIILDNAGFELVTDLVLADFLISSGLAKQIRFHGKSIPWFVSDVTKQDFEWTIKQTMAANHKWMSASGVQWKHFMKEGTWSYHDHPFWTLPHEFCDMTVDAANLYSTLQTSDLILFKGDLNYRKLTGDRKWEHTVRFDQALRGFQPAPLCSLRTLKADVQVGLQAGHAEKLSTQDPDWMTNGKYAVVQFSSPHREQ

SEQ ID NO: 16 - protein, 436 aa, D. melanogaster
  misc_feature (1)..(436): D. melanogaster (24657549)
MGSETDFDAKNGIVDGPTPPHTELAALYKQSFAYYTFRVRLPSTLATIADSLVKDKDVLLATYGAAAEADIEQTTKEVRQLRDDILSNGPLLPFGENDSDSEVWNAFLEKLPKEKRTYFSVCWLYAECYMYRKISSIFRATAHLAAYDYFSQQKQTATKLSVDAMLAVAKATRHNERNSDTFRQLIKLNLWGNRCDLSITSGKQVKPTGNAFDQVTDLEEKLLIDGTAEVWKALDGASGEGIVDIIFDNAGYELYTDLILAEYIIDKGLAAKVRFNPKAIPWFISDVMEHDFHWALQFLADHPDPVLSEVGKKWQRLTTEGKFELSPLEHFWTSPYEFYRMPEVNPSLYDRLKEAQLVIFKGDLNYRKLLGDFSWDSTESFETCLRGFRPSNLCTLRTIKADLICGLGAGVADQLFAKDKEWMLTGEYGVIQFASK

SEQ ID NO: 17 - protein, 450 aa, C. elegans
  misc_feature (1)..(450): C. elegans (17560374)
MENADEYDHLAPKLRGKKEGTFAYYTVRDRWPKIVTGLVDQLAQKRASLIEKYGSEVESDIAAILEVFSKLRYEIMTDKPLCNLMDTQLDSEMWRNLLSDMRTAAMPDEVEDLTFFKGPWLFVECWLYRFIWSTFAKTIRLSEYDYFQDSKRKNFLDHLPQIEESAAFINKISAKDAPVHELFGINTILKMSLWGNRADMSLTGGDDHTLAMSSMSASSKLADFVLIDDVNDMIVKVLGPLKINANHETNRRIDIILDNSGVELTGDLIVAEFFISRGFADKVVIHGKAIPWFVSDVTKPDLDWTIEQLKNGENIGEESRALGEKLEKRMKSGQIVYQDHLFWISPHAYYAMEKEARDLYDDLKNSSLIIFKGDLNYRKLVGDRDWDLDTSFKTACRGFAPCPFMALRTLKAETVAGLSEESIAILLEKFEEDNTWMTSGEYAVCQLGGI

SEQ ID NO: 18 - protein, 442 aa, S. pombe [sequence omitted]

SEQ ID NO: 19 - protein, 470 aa, S. cerevisiae
  misc_feature (1)..(470): S. cerevisiae (6323669)
MTIPGRFMTIDKGTFGEYTASTRWPIIIQNAIDDLSKHQETEKSNGTKFEQGEVIKKELKEFRQEIIDRVPLRPFTEEEIKIANVPLSFNEYLKKHPEVNWGAVEWLFSEVYLYRRVNVLFQRQCEWAKFDIFNRLKQSTFESSFYGVVELALRYENLLPQLREMKQNPGNEIDDILKVLFKEFIEISLWGNATDLSLLTNATLEDIKSIQGAKARAASESKIVVNDTEKAWEVLTKARADANSREIRVDFVLDNSGFELYADLMLAAFLLQSGLATKCIFHAKDIPYMVSDVMLKDFDILVHDLRDREFFPSGEPSTKESRALDLFAGEMEKFVSSGKIEFREDSFWTTELDYWNLDANETKYHGSILHKDLQKSNLVIFKGDLNYRKLTGDRKWPRTTKWETAIGPLATNGITSLSLRTCKADVQVALPEGLDAKLSQEWEKENPGRGSWWCCSGKWAVICFCSGIHK

SEQ ID NO: 20 - protein, 261 aa, Artificial Sequence
  Synthetic - Spot 1
MFEARLVQGSILKKVLEALKDLINEACWDISSSGVNLQSMDSSHVSLVQLTLRSEGFDTYRCDRNLAMGVNLTSMSKILKCAGNEDIITLRAEDNADTLALVFEAPNQEKVSDYEMKLMDLDVEQLGIPEQEYSCVVKMPSGEFARICRDLSHIGDAVVISCAKDGVKFSASGELGNGNIKLSQTSNVDKEEEAVTIEMNEPVQLTFALRYLNFFTKATPLSSTVTLSMSADVPLVVEYKIADMGHLKYYLAPKIEDEEGS

SEQ ID NO: 21 - protein, 261 aa, Artificial Sequence; identical to SEQ ID NO: 20
  Synthetic - Spot 2

SEQ ID NO: 22 - protein, 261 aa, Artificial Sequence; identical to SEQ ID NO: 20
  Synthetic - Spot 3

SEQ ID NO: 23 - protein, 261 aa, Artificial Sequence; identical to SEQ ID NO: 20
  Synthetic - Spot 4

SEQ ID NO: 24 - protein, 261 aa, Artificial Sequence; identical to SEQ ID NO: 20
  Synthetic - Spot 5

SEQ ID NO: 25 - protein, 261 aa, Artificial Sequence; identical to SEQ ID NO: 20
  Synthetic - Spot 6